(19) United States
(12) Patent Application Publication
(10) Pub. No.: US 2004/0229777 A1
(43) Pub. Date: Nov. 18, 2004

(54) Crystalline phases of a potent HCV inhibitor

(75) Inventors: Michael Kenneth Cerreta, Newtown, CT (US); John Andrew Smoliga, Brookfield, CT (US); Richard J. Varsolona, Scotch Plains, NJ (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim International GmbH, Ingelheim (DE)

(21) Appl. No.: 10/809,597

(22) Filed: Mar. 25, 2004

Related U.S. Application Data
(60) Provisional application No. 60/458,188, filed on Mar. 27, 2003.

(51) Int. Cl. A61K 38/12; C07K 5/12; A61K 31/4709

(52) U.S. Cl. 514/10; 514/312; 530/317

(57) ABSTRACT

This invention relates to novel crystalline phases of the following Compound (1), methods for the preparation thereof, pharmaceutical compositions thereof, and their use in the treatment of Hepatitis C Viral (HCV) infection:

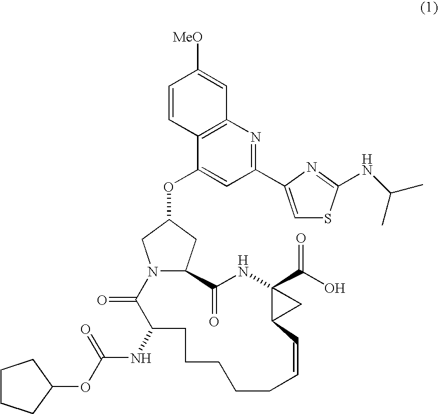

This application claims benefit to U.S. Provisional Application No. 60/458,188, filed Mar. 27, 2003, which application is herein incorporated by reference in its entirety.

### FIELD OF THE INVENTION

This invention relates to novel crystalline phases of Compound (1) described herein, methods for the preparation thereof, pharmaceutical compositions thereof, and their use in the treatment of Hepatitis C Viral (HCV) infection.

### BACKGROUND OF THE INVENTION

The following Compound (1):

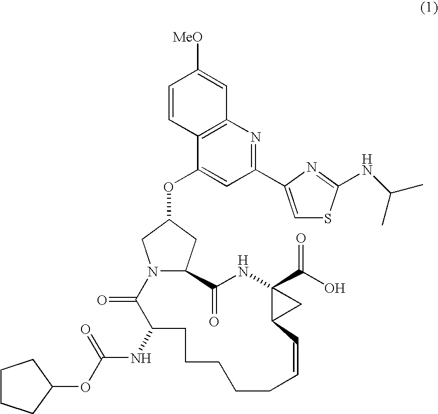

having the chemical name: cyclopropa[e]pyrrolo[1,2-a][1,4]diazacyclopentadecine-14a(5H)-carboxylic acid, 6-[[(cyclopentyloxy)carbonyl]amino]-1,2,3,6,7,8,9,10,11,13a, 14,5,16,16a-tetradecahydro-2-[[7-methoxy-2-[2-[(1-methylethyl)amino]-4-thiazolyl]-4-quinolinyl]oxy]-5,16-dioxo-, (2R,6S,12Z,13aS,14aR,16aS)-, (Registry No. 300832-84-2, is known as a selective and potent inhibitor of the HCV NS3 serine protease. Compound (1) falls within the scope of the macrocyclic peptide series of HCV inhibitors disclosed in WO 00/59929 (Boehringer Ingelheim (Canada) Ltd.). Compound (1) is disclosed specifically as Compound # 822 in WO 00/59929, and its method of synthesis is described therein, as well as its utility in the treatment and prevention of HCV infection via its ability to inhibit HCV NS3 serine protease.

The synthetic procedure described in WO 00/59929 results in the formation of Compound (1) as an amorphous solid which is a form that is generally less suitable for full-scale pharmaceutical processing. Thus, there is a need to produce Compound (1) in a crystalline form to enable formulations to meet exacting pharmaceutical requirements and specifications. Furthermore, the process by which Compound (1) is produced needs to be one which is amenable to large-scale production. Additionally, it is desirable that the product should be in a form that is readily filterable and easily dried. Finally, it is economically desirable that the product be stable for extended periods of time without the need for specialized storage conditions.

We have now surprisingly and unexpectedly found that Compound (1) can be prepared in crystalline phase. Thus, the present invention provides Compound (1) in new crystalline phases designated herein as Types A and B.

### SUMMARY OF THE INVENTION

The present inventors have discovered two novel crystalline phases of Compound (1), referred to hereinafter as Types A and B. Types A and B are both believed to be nonstoichiometric variable hydrates, and like the other phases of the compound, are useful in the treatment of HCV infection.

Type A exhibits a characteristic X-ray powder diffraction (XRPD) pattern with characteristic peaks expressed in degrees 2θ (±0.4 degrees 2θ) at 6.9, 8.0, 12.5, 13.9, 14.9, 16.1, 16.7, 17.5, 20.9, 22.7 and 24.1 measured using CuKα radiation. In particular, the peak expressed at 6.9 degrees 2θ (±0.4 degrees 2θ) is the most intense XRPD peak for Type A in that all other peaks in the pattern have less than 75% intensity relative to this peak, and this is sufficient to characterize and distinguish Type A from Type B.

Type A also is hygroscopic and absorbs water up to a maximum of about 3.7% by weight.

Type B exhibits a characteristic X-ray powder diffraction (XRPD) pattern with characteristic peaks expressed in degrees 2θ (±0.2 degrees 2θ) at 5.4, 6.7, 9.4, 10.3, 10.9, 11.6, 13.2 and 20.9 measured using CuKα radiation at a relative humidity level of about 20% to 40% and at a temperature of about 20 to 25° C. In particular, the peak expressed at 5.4 degrees 2θ (±0.2 degrees 2θ) is unique to Type B and is sufficient to characterize and distinguish Type B from Type A.

Type B also is hygroscopic and absorbs water up to a maximum of about 4.5% by weight and readily loses water resulting in a partial collapse of its crystal structure when exposed to low relative humidity (e.g., dry nitrogen) and/or elevated temperature (˜90° C.).

Another embodiment is directed to a process for preparing Type A, said process comprising the following steps (i) and either (ii)(a) or (ii)(b):

(i) dissolving Compound (1) in an aliphatic alcohol solvent optionally containing water as a co-solvent; and

(ii)(a) adding water, or a mixture of water and an aliphatic alcohol, to the solution obtained in step (i) while maintaining the solution at a temperature above about 55° C.; or

(ii)(b) adding the solution obtained in step (i) to water, or a mixture of water and an aliphatic alcohol, while maintaining the water, or mixture of water and an aliphatic alcohol, at a temperature above about 55° C.

Another embodiment is directed to an alternative process for preparing Type A, said process comprising the following steps:

(i) dissolving or suspending Compound (1) in acetonitrile to form a solution or slurry;

(ii) optionally seeding the solution or slurry obtained in step (i) with Type A;

(iii) heating the solution or slurry to a temperature of at least about 75° C.;

(iv) adding water to the heated solution or slurry obtained in step (iii) while maintaining the solution or slurry at a temperature of at least about 75° C. to obtain a solution or slurry having a water content of about 3 to 5 weight percent; and

(v) slowly cooling the solution or slurry obtained in step (iv).

Another embodiment is directed to a crystalline phase of Compound (1) prepared by a process described above.

Another embodiment is a process for preparing Type B, said process comprising the following steps (i) and (ii), or steps (i)(a) and (ii)(a):

(i) dissolving Compound (1) in a suitable solvent by heating a mixture of Compound (1) and the solvent; and

(ii) cooling the solution obtained in step (i); or

(i)(a) dissolving Compound (1) in an aliphatic alcohol solvent; and

(ii)(a) evaporating the aliphatic alcohol solvent from the solution obtained in step (i)(a).

Another embodiment is directed to a crystalline phase of Compound (1) prepared by a process described above.

In each of the aforementioned methods of preparing crystalline phases of Compound (1), the crystals formed may be recovered by any method known in the art.

Yet another embodiment is directed to mixtures of Types A and B.

Yet another embodiment is directed to a pharmaceutical composition comprising Type A or B of Compound (1), or a mixture thereof, and at least one pharmaceutically acceptable carrier or diluent.

Yet another embodiment is directed to a method of treating HCV infection in a mammal comprising administering to said mammal a therapeutically effective amount of Types A or B of Compound (1), or a mixture thereof.

### BRIEF DESCRIPTION OF THE DRAWINGS

FIG. 1 is a characteristic X-ray Powder Diffraction (XRPD) pattern for Type A at a relative humidity of ˜30%.

FIG. 2 is a characteristic X-ray Powder Diffraction (XRPD) pattern for Type A at a relative humidity of ˜85%.

FIG. 3 is a water adsorption/desorption isotherm of Type A at 25° C.

FIG. 4 is the DSC thermal curve for Type A crystals prepared by an ethanol/water process, where the DSC is performed at a heating rate of 10° C. per minute.

FIG. 5 is the DSC thermal curve for Type A crystals prepared by an acetonitrile process, where the DSC is performed at a heating rate of 10° C. per minute.

FIG. 6 is a characteristic X-ray Powder Diffraction (XRPD) pattern for Type B at a relative humidity of ˜30%.

FIG. 7 is a water adsorption/desorption isotherm of Type B at 25° C.

### DETAILED DESCRIPTION OF THE INVENTION

Definitions

Terms not specifically defined herein should be given the meanings that would be given to them by one of skill in the art in light of the disclosure and the context. As used throughout the present application, however, unless specified to the contrary, the following terms have the meaning indicated:

The term “Type A” means a crystalline phase of Compound (1) that has at least the following characteristic:

an X-ray powder diffraction pattern having at least a characteristic peak at 6.9 degrees 2θ (±0.4 degrees 2θ) measured using CuKα radiation,wherein all other peaks in the pattern have less than 75% intensity relative to the peak at 6.9 degrees 2θ (±0.4 degrees 2θ).

The term “Type B” means a crystalline phase of Compound (1) that has at least the following characteristic:

an X-ray powder diffraction pattern having at least a characteristic peak at 5.4 degrees 2θ (±0.2 degrees 2θ) measured using CuKα radiation at a relative humidity level of about 20% to 40% at a temperature of about 20 to 25° C.;

The term “about” means within 5%, and more preferably within 1% of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “treating” with respect to the treatment of a disease-state in a patient include:

(i) inhibiting or ameliorating the disease-state in a patient, e.g., arresting or slowing its development; or

(ii) relieving the disease-state in a patient, i.e., causing regression or cure of the disease-state.

Type A of Compound (1)

Type A is a variable hydrate crystalline phase of Compound (1), i.e., the number of water molecules associated with each molecule of Compound (1) may vary. The term “hydrate” refers to a crystal form of Compound (1) wherein at least one molecule of Compound (1) in the crystal is associated with water. The number of water molecules associated with each molecule of Compound (1) in Type A can vary from 0 to about 2, i.e. Type A can be anhydrous or a hydrate and all such forms and levels of hydration of Type A are contemplated within the scope of the present invention. For example, Type A can be anhydrous or a monohydrate or hemihydrate of Compound (1). The term “monohydrate” as used herein refers to a hydrate in which one molecule of water is associated with each molecule of Compound (1). The term “hemihydrate” as used herein refers to a hydrate in which one molecule of water is associated with two molecules of Compound (1).

Analyses of Type A by XRPD under various relative humidity conditions (dry nitrogen up to ˜85% RH) and elevated temperature (up to 160° C.) indicate that the crystal lattice expands at higher RH levels while maintaining its overall structure and contracts when exposed to ambient conditions. This behavior is typical of channel hydrates, whereby water resides within the channels in the crystal lattice and can readily move in and out of the structure. Type A is therefore believed to be a type of channel hydrate.

In general, Type A exhibits a characteristic X-ray powder diffraction (XRPD) pattern with characteristic peaks expressed in degrees 2θ (±0.4 degrees 2θ) at 6.9, 8.0, 12.5, 13.9, 14.9, 16.1, 16.7, 17.5, 20.9, 22.7 and 24.1. In particular, the peak expressed at 6.9 degrees 2θ (±0.4 degrees 2θ) is the most intense XRPD peak for Type A in that all other peaks in the pattern have less than 75% intensity relative to this peak, and this characteristic is unique to type A.

The XRPD pattern of Type A varies slightly with its moisture content in that there is a slight shifting of the pattern at different relative humidity levels. For example, in the range of low RH (about 2%) to high RH (about 85%) the shift of the pattern is about ±0.2 degrees 2θ from the pattern at ambient RH (in general, a low RH results in a positive shift, whereas a high RH results in a negative shift). The XRPD of Type A is therefore defined herein including an “error” range (±0.4 degrees 2θ) believed sufficient to cover the XRPD pattern of Type A at all RH levels. The present invention is intended to cover Type A at all RH levels.

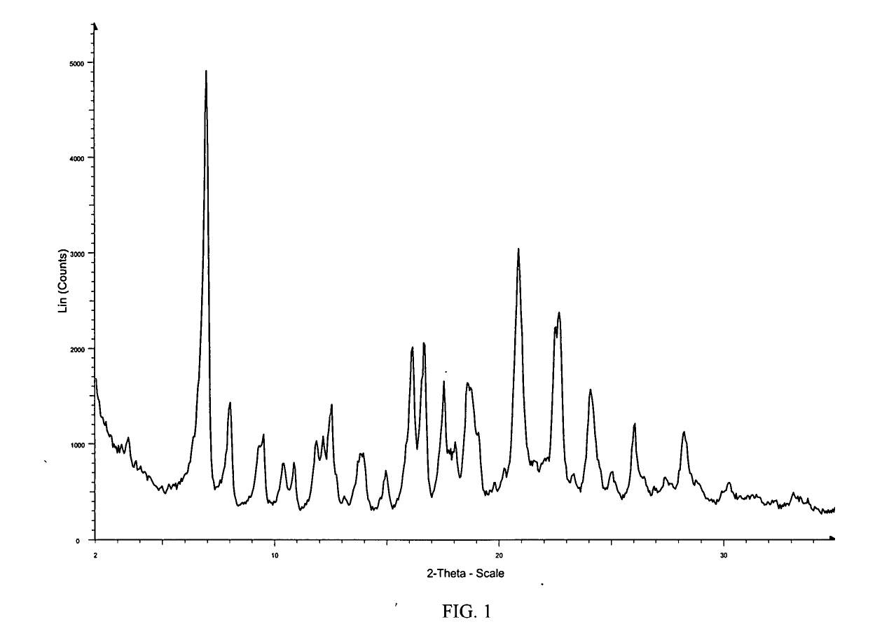

The XRPD pattern of Type A at a relative humidity of ˜30% is shown in FIG. 1. The characteristic peak positions and relative intensities for the XRPD pattern in FIG. 1 is shown in Table 1 below.

TABLE 1
Compound (1)
Type A Hydrate @ ˜30% RH

| Angle | |
|---|---|
| 2-Theta° | Rel. Intensity % |
| 6.9 | 100 |
| 8 | 30 |
| 12.5 | 29 |
| 13.9 | 18 |
| 14.9 | 15 |
| 16.1 | 41 |
| 16.7 | 42 |
| 17.5 | 34 |
| 20.9 | 62 |
| 22.7 | 49 |
| 24.1 | 32 |

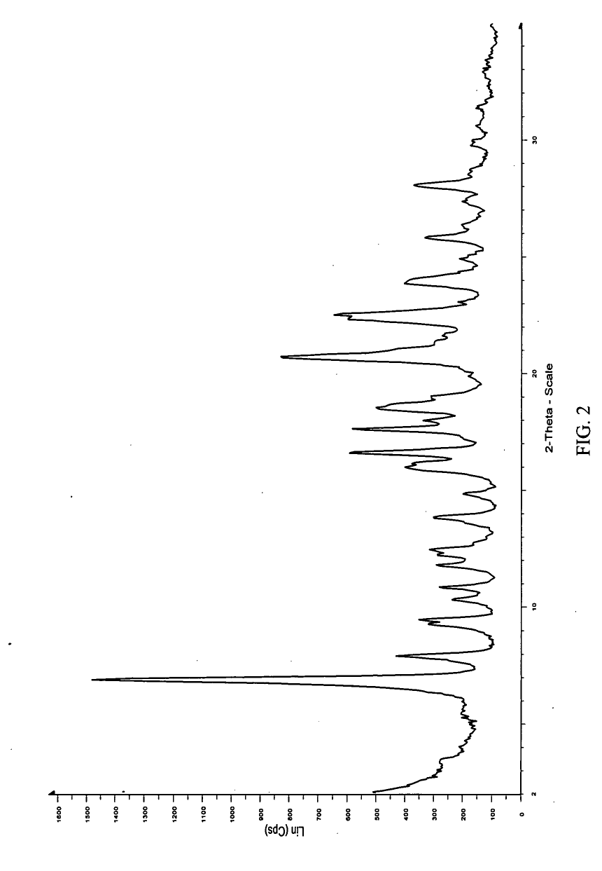

The XRPD pattern of Type A at a relative humidity of ˜85% is shown in FIG. 2. The characteristic peak positions and relative intensities for the XRPD pattern in FIG. 2 is shown in Table 2 below.

TABLE 2
Compound (1)
Type A Hydrate @ ˜85% RH

| Angle | |
|---|---|
| 2-Theta° | Rel. Intensity % |
| 6.9 | 100 |
| 7.9 | 30 |
| 12.5 | 21 |
| 13.8 | 20 |
| 14.8 | 13 |
| 16 | 27 |
| 16.6 | 40 |
| 17.7 | 39 |
| 20.7 | 56 |
| 22.6 | 44 |
| 23.9 | 27 |

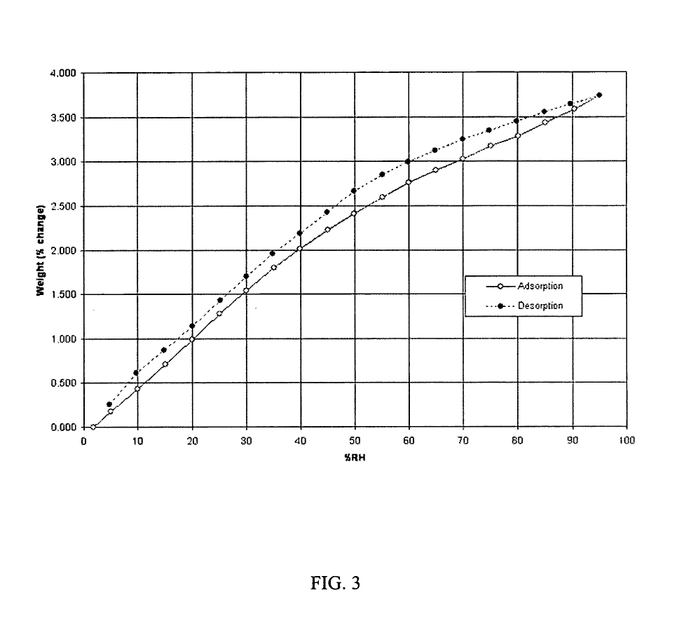

FIG. 3 shows the water adsorption/desorption curves for Type A at 25° C. It is clear from FIG. 3 that the moisture content of Type A varies depending on the relative humidity of its environment, up to a maximum water sorption level of about 3.7% by weight.

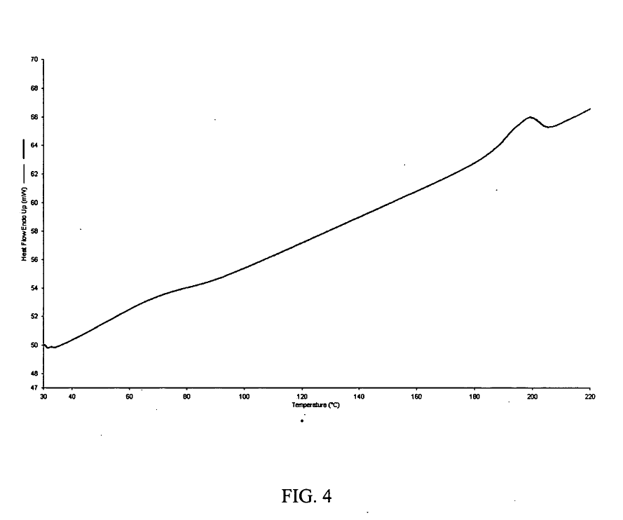

FIG. 4 shows the Differential Scanning Calorimetry (DSC) thermal curve for Type A crystals prepared by the ethanol/water process of Example 1, where the DSC is performed at a heating rate of 10° C. per minute.

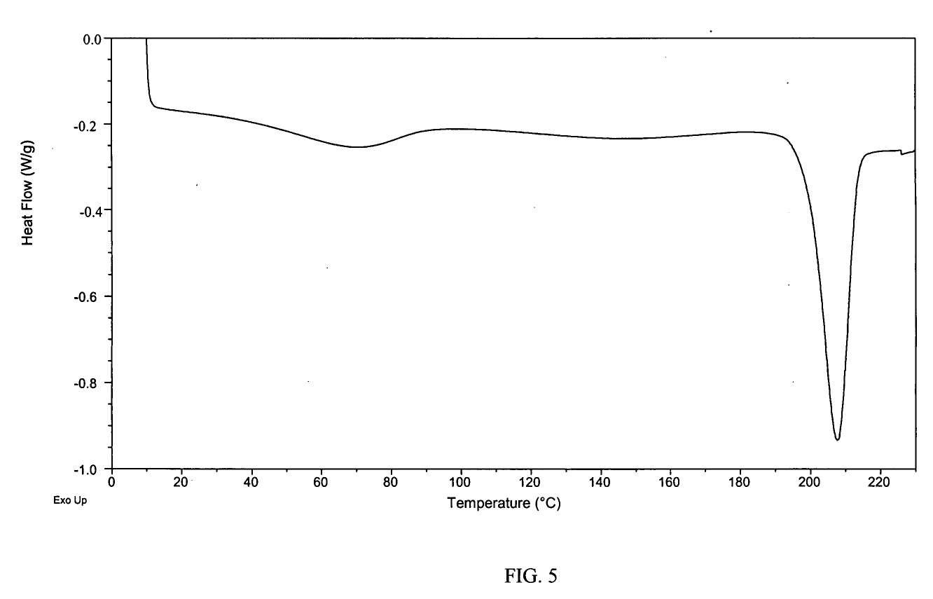

FIG. 5 shows the Differential Scanning Calorimetry (DSC) thermal curve for Type A crystals prepared by the acetonitrile process of Example 2, where the DSC is performed at a heating rate of 10° C. per minute.

Accordingly, in one embodiment the present invention is directed to a crystalline phase of Compound (1) that has at least the following characteristic:

an X-ray powder diffraction pattern having at least a characteristic peak at 6.9 degrees 2θ (±0.4 degrees 2θ) measured using CuKα radiation, wherein all other peaks in the pattern have less than 75% intensity relative to the peak at 6.9 degrees 2θ (±0.4 degrees 2θ).

Another embodiment is directed to a crystalline phase of Compound (1) having an XRPD pattern with a characteristic peak at 6.9 degrees 2θ (±0.4 degrees 2θ) as described above and having additional characteristic peaks at least at 20.9 and 22.7 degrees 2θ (±0.4 degrees 2θ) measured using CuKα radiation.

Another embodiment is directed to a crystalline phase of Compound (1) having an XRPD with a characteristic peak at 6.9 degrees 2θ (±0.4 degrees 2θ) as described above and having additional characteristic peaks at least at 16.1, 16.7, 20.9 and 22.7 degrees 2θ (±0.4 degrees 2θ) measured using CuKα radiation.

Another embodiment is directed to a crystalline phase of Compound (1) having an XRPD with a characteristic peak at 6.9 degrees 2θ (±0.4 degrees 2θ) as described above and having additional the characteristic peaks at least at 8.0, 12.5, 13.9, 14.9, 16.1, 16.7, 17.5, 20.9, 22.7 and 24.1 degrees 2θ (±0.4 degrees 2θ) measured using CuKα radiation.

Another embodiment is directed to a crystalline phase of Compound (1) exhibiting an XRPD pattern substantially the same as that shown in FIG. 1 at a relative humidity of about 30%.

Another embodiment is directed to a crystalline phase of Compound (1) exhibiting an XRPD pattern substantially the same as that shown in FIG. 2 at a relative humidity of about 85%

Another embodiment is directed to a crystalline phase of Compound (1) having an XRPD with a characteristic peak at 6.9 degrees 2θ (±0.4 degrees 2θ) as described above and also exhibiting a water adsorption/desorption isotherm substantially the same as that shown in FIG. 3 at 25° C.

Another embodiment is directed to a crystalline phase of Compound (1) having an XRPD with a characteristic peak at 6.9 degrees 2θ (±0.4 degrees 2θ) as described above and also exhibiting a DSC thermal curve substantially the same as that shown in FIG., at a heating rate of 10° C. per minute.

Another embodiment is directed to a crystalline phase of Compound (1) having an XRPD with a characteristic peak at 6.9 degrees 2θ (±0.4 degrees 2θ) as described above and also exhibiting a DSC thermal curve substantially the same as that shown in FIG. 5, at a heating rate of 10° C. per minute.

Another embodiment is directed to a Compound (1) wherein at least 95%, more preferably at least 99%, of said substance is present in the form of Type A crystalline phase.

The present invention provides a process for the preparation of Type A which comprises crystallizing Compound (1) from a solution in solvents under conditions which yield Type A. The precise conditions under which Type A is formed may be empirically determined and it is only possible to give methods which have been found to be suitable in practice.

It has been found that Type A of Compound (1) may be prepared by a process comprising the following steps (i) and either (ii)(a) or (ii)(b):

(i) dissolving Compound (1) in an aliphatic alcohol solvent optionally containing water as a co-solvent; and

(ii)(a) adding water, or a mixture of water and an aliphatic alcohol, to the solution obtained in step (i) while maintaining the solution at a temperature above about 55° C., preferably above about 70° C.; or

(ii)(b) adding the solution obtained in step (i) to water, or a mixture of water and an aliphatic alcohol, while maintaining the water, or the mixture of water and an aliphatic alcohol, at a temperature above about 55° C., preferably above about 70° C.

Aliphatic alcohols that may be employed in various steps of this process include, for example, ethanol (e.g., denatured, 200 proof or 100% pure), isopropanol, methanol and butanol, preferably ethanol. The resulting crystals of Type A may be recovered by any conventional methods known in the art.

In one preferred embodiment, amorphous Compound (1) is dissolved in an aliphatic alcohol solvent (e.g., ethanol), containing up to about 10% v/v water as co-solvent, by stirring and heating the mixture until Compound (1) completely dissolves. A separate water addition solution is prepared containing water and up to about 10% v/v aliphatic alcohol (e.g., ethanol), and this water addition solution is added approximately linearly over time to the Compound (1) solution while maintaining the mixture at a temperature above about 60° C., preferably above about 70° C. Type A of Compound (1) begins to crystallize during the addition of the water solution. The resulting crystal slurry is cooled and stirred, and the crystals are then filtered, washed and dried using conventional techniques.

It has been found that Type A may also be prepared by an alternative process comprising the following steps:

(i) dissolving or suspending Compound (1) in acetonitrile to form a solution or slurry;

(ii) optionally seeding the solution or slurry obtained in step (i) with Type A;

(iii) heating the solution or slurry to a temperature of at least about 75° C.;

(iv) adding water to the heated solution or slurry obtained in step (iii) while maintaining the solution or slurry at a temperature of at least about 75° C. to obtain a solution or slurry having a water content of about 3 to 5 weight percent; and

(v) slowly cooling the solution or slurry obtained in step (iv).

The Compound (1) used as the starting material to be dissolved or suspended in the acetonitrile can be Type A, Type B or the amorphous form of Compound (1). The solution may then be optionally seeded with Type A crystals using conventional seeding techniques. Prior to the addition of water, the solution is heated to a temperature of at least about 75° C., preferably for about 45 minutes. Water is then added to obtain a solution having a water content of about 3 to 5 weight percent, preferably about 4 weight percent. The solution is then slowly cooled, preferably at a cooling rate of about 6 to 10° C./hr, for example at about 8° C./hr. The Type A form of Compound (1) begins to crystallize upon cooling the solution. The resulting crystals of Type A may be recovered (e.g., filtered, washed and dried) by any conventional method known in the art. The process steps may of course be facilitated by conventional agitation techniques, e.g., stirring, and other conventional techniques as would be well understood.

It has been found that the Type A crystals preparing using this alternative acetonitrile technique have improved crystallinity, e.g., resulting in considerable crystal growth which in turn greatly enhances filtration rates and results in an isolated product with an increase in the crystalline nature of the isolated phase, Type A.

Type B of Compound (1)

Type B is also a variable hydrate crystalline phase of Compound (1), i.e., the number of water molecules associated with each molecule of Compound (1) may vary. However, unlike Type A, Type B readily loses water resulting in a partial collapse of its crystal structure when exposed to low relative humidity (e.g., dry nitrogen) and/or elevated temperature (˜90° C.). For this reason, it is preferable to maintain Type B at a modest RH level of about 20% to 40%.

Type B exhibits a characteristic X-ray powder diffraction (XRPD) pattern with characteristic peaks expressed in degrees 2θ (±0.2 degrees 2θ) at 5.4, 6.7, 9.4, 10.3, 10.9, 11.6, 13.2 and 20.9 measured using CuKα radiation at a relative humidity level of about 20% to 40% and at a temperature of about 20 to 25° C. In particular, the peak expressed in degrees 2θ (±0.2 degrees 2θ) at 5.4 is unique to Type B and is sufficient to characterize and distinguish Type B from Type A.

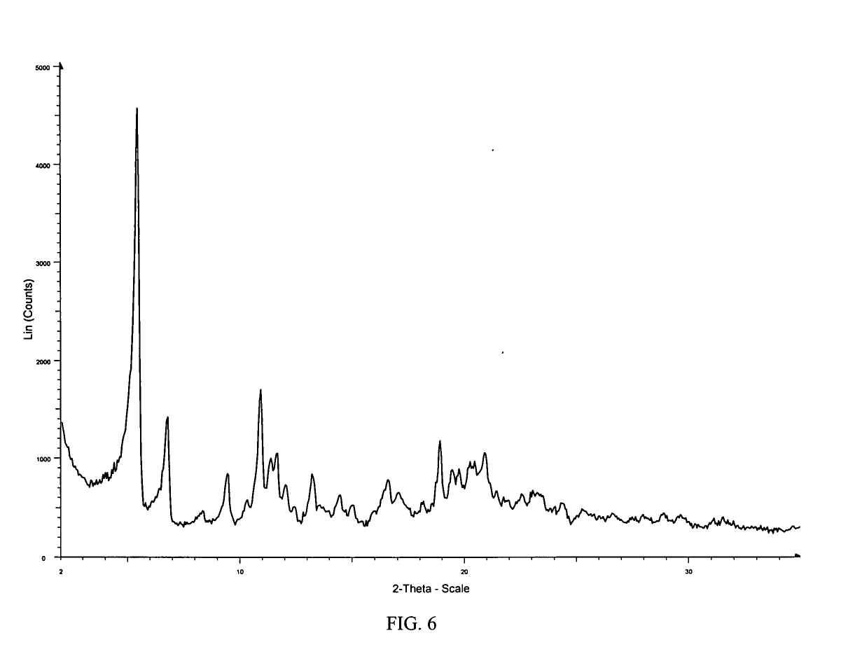

The XRPD pattern of Type B at a relative humidity of ˜30% is shown in FIG. 6. The characteristic peak positions and relative intensities for the XRPD pattern in FIG. 6 is shown in Table 3 below.

TABLE 3
Compound (1)
Type B Hydrate @ ˜30% RH

| Angle | |
|---|---|
| 2-Theta° | Rel. Intensity % |
| 5.4 | 100 |
| 6.7 | 31 |
| 9.4 | 19 |
| 10.3 | 13 |
| 10.9 | 37 |
| 11.6 | 23 |
| 13.2 | 18 |
| 20.9 | 23 |

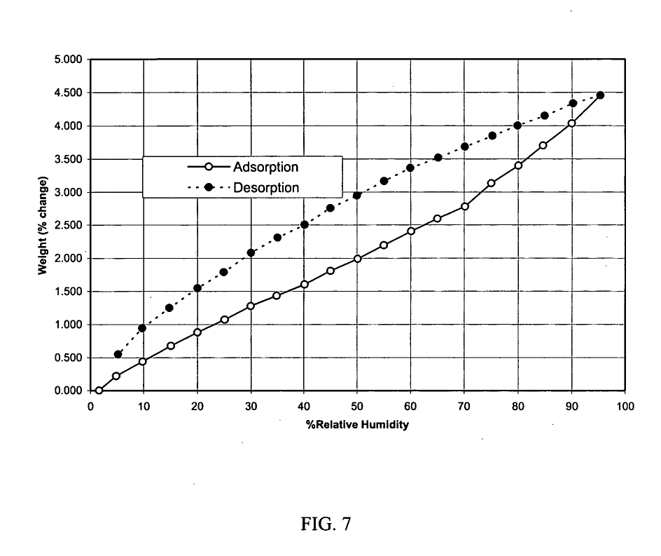

FIG. 7 shows the water adsorption/desorption curves for Type B at 25° C. It is clear from FIG. 7 that the moisture content of Type B varies depending on the relative humidity of its environment, up to a maximum water sorption level of about 4.5% by weight.

Accordingly, in one embodiment the present invention is directed to a crystalline phase of Compound (1) that has at least the following characteristic:

an X-ray powder diffraction pattern having at least a characteristic peak at 5.4 degrees 2θ (±0.2 degrees 2θ) measured using CuKα radiation at a relative humidity level in the range of about 20% to 40% and at a temperature of about 20 to 25° C.

Another embodiment is directed to a crystalline phase of Compound (1) having an XRPD with the characteristic peaks at least at 5.4, 6.7 and 10.9 expressed in degrees 2θ (±0.2 degrees 2θ) measured using CuKα radiation at a relative humidity level of about 20% to 40% and at a temperature of about 20 to 25° C.

Another embodiment is directed to a crystalline phase of Compound (1) having an XRPD with the characteristic peaks at least at 5.4, 6.7, 10.9, 11.6 and 20.9 expressed in degrees 2θ (±0.2 degrees 2θ) measured using CuKα radiation at a relative humidity level of about 20% to 40% and at a temperature of about 20 to 25° C.

Another embodiment is directed to a crystalline phase of Compound (1) having an XRPD with the characteristic peaks at least at 5.4, 6.7, 9.4, 10.3, 10.9, 11.6, 13.2 and 20.9 expressed in degrees 2θ (±0.2 degrees 2θ) measured using CuKα radiation at a relative humidity level of about 20% to 40% and at a temperature of about 20 to 25° C.

Another embodiment is directed to a crystalline phase of Compound (1) exhibiting an XRPD pattern substantially the same as that shown in FIG. 6 at a relative humidity of about 30%.

Another embodiment is directed to a crystalline phase of Compound (1) having an X-ray powder diffraction pattern having at least a characteristic peak at 5.4 degrees 2θ (±0.2 degrees 2θ) measured using CuKα radiation at a relative humidity level in the range of about 20% to 40% and at a temperature of about 20 to 25° C., and also exhibiting a water adsorption/desorption isotherm substantially the same as that shown in FIG. 7 at 25° C.

Another embodiment is directed to a Compound (1) wherein at least 95%, more preferably at least 99%, of said substance is present in the form of Type B crystalline phase.

The present invention provides a process for the preparation of Type B which comprises crystallizing Compound (1) from a solution in solvents under conditions which yield Type B. The precise conditions under which Type B is formed may be empirically determined and it is only possible to give methods which have been found to be suitable in practice.

It has been found that Type B of Compound (1) may be prepared by a process comprising:

(i) dissolving Compound (1) in a suitable solvent by heating a mixture of Compound (1) and the solvent; and

(ii) cooling the solution obtained in step (i).

Suitable solvents that may be used in step (i) include, for example, aliphatic alcohols such as ethanol (e.g., denatured, 200 proof or 100% pure), isopropanol, methanol and butanol, as well as ethyl acetate. The mixture of Compound (1) in the solvent is heated until the Compound (1) solids dissolve. The dissolution temperature will, of course, depend on the solvent. When ethanol is used, the dissolution occurs at about 42° C., but when other solvents are used the dissolution temperature may be higher. Type B of Compound (1) begins to crystallize upon cooling the solution. Anti-solvents, such as water or heptane, may be added to the solution prior to or during crystallization to increase the yield.

Type B of Compound (1) may also be prepared by a process comprising:

(i) dissolving Compound (1) in an aliphatic alcohol solvent; and

(ii) evaporating the aliphatic alcohol solvent from the solution obtained in step (i).

Aliphatic alcohols that may be employed in step (i) include, for example, ethanol (e.g., denatured, 200 proof or 100% pure), isopropanol, methanol and butanol, preferably ethanol. Type B of Compound (1) begins to crystallize upon evaporation of the solution obtained in step (i). Evaporation can be by slow or fast evaporation methods known in the art. One preferred method of fast evaporation involves removing the solvent quickly such as by vacuum. One preferred method of slow evaporation involves incubating the mixture at room temperature to allow evaporation to occur slowly.

In either method above, the resulting crystals of Type B may be recovered (e.g, filtered, washed and dried) by any conventional methods known in the art.

Mixtures

Another embodiment of the present invention is directed to mixtures of Types A and B. Such mixtures may be prepared, for example, by physically mixing together the two types of crystals, each prepared as described previously, using conventional techniques. These mixtures are typically characterized by an XRPD pattern having the peaks characteristic for Type A and also the peaks characteristic for B. As described herein, such mixtures can be used in the pharmaceutical compositions and methods of treatment according to the present invention.

Another embodiment is directed to a Compound (1) wherein at least 50%, preferably at least 75%, more preferably at least 90%, of said substance is present in the form of Type A or Type B, or a mixture thereof. The presence of such amounts of Types A or B, or mixtures thereof, in a quantity of Compound (1) is typically measurable using XRPD analysis of the compound.

Pharmaceutical Compositions and Methods

The aforementioned crystal phases of Compound (1) are useful as anti-HCV agents in view of the inhibitory activity of Compound (1) against HCV NS3 serine protease. Types A and B, and mixtures thereof, are therefore useful in treatment of HCV infection in a mammal. The appropriate dosage amounts and regimens for a particular patient can be determined by methods known in the art and by reference to the disclosure in WO 00/59929.

Generally, a therapeutically effective amount for the treatment of HCV infection in the mammal is administered. In one embodiment, about 50mg to 1000mg is administered per adult human per day in single or multiple doses.

Specific optimal dosage and treatment regimens for any particular patient will of course depend upon a variety of factors, including the age, body weight, general health status, sex, diet, time of administration, rate of excretion, drug combination, the severity and course of the infection, the patient's disposition to the infection and the judgment of the treating physician. Generally, treatment is initiated with small dosages substantially less than the optimum dose. Thereafter, the dosage is increased by small increments until the optimum effect under the circumstances is reached. In general, the compound is most desirably administered at a concentration level that will generally afford antivirally effective results without causing any harmful or deleterious side effects.

Types A or B, or a mixture thereof, at a selected dosage level is typically administered to the patient via a pharmaceutical composition. See, e.g., the description in WO 00/59929 for the various types of compositions that may be employed in the present invention. The pharmaceutical composition may be administered orally, parenterally or via an implanted reservoir. The term parenteral as used herein includes subcutaneous, intracutaneous, intravenous, intramuscular, intra-articular, intrasynovial, intrasternal, intrathecal, and intralesional injection or infusion techniques. Oral administration or administration by injection are preferred.

The pharmaceutical compositions of this invention may contain any conventional non-toxic pharmaceutically-acceptable carriers, diluents, adjuvants, excipients or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example. Tween 80) and suspending agents.

The pharmaceutical compositions may also be in the form of an oral pharmaceutical composition comprising Type A, Type B, or a mixture thereof, and at least one pharmaceutically acceptable carrier or diluent. The oral pharmaceutical compositions may be orally administered in any orally acceptable dosage form including, but not limited to, tablets, capsules (e.g., hard or soft gelatin capsules), and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. Examples of soft gelatin capsules that can be used include those disclosed in EP 649651 B1 and U.S. Pat. No. 5,985,321. When aqueous suspensions are administered orally, the active ingredient is combined with emulsifying and suspending agents. If desired, certain sweetening and/or flavoring and/or coloring agents may be added.

Other suitable vehicles or carriers for the above noted formulations and compositions can be found in standard pharmaceutical texts, e.g. in “Remington's Pharmaceutical Sciences”, 19^{th} ed., Mack Publishing Company, Easton, Pa., 1995.

Methods of Characterization

1. X-Ray Powder Diffraction

X-ray powder diffraction analyses were conducted on a Bruker AXS X-Ray Powder Diffractometer Model D8 Advance, available from Bruker AXS, Inc. of Madison, Wis., using CuKα radiation. The instrument is equipped with a long fine focus x-ray tube. The tube power was set to 40 kV and 30 mA. The instrument was operated in parallel beam mode with a Gobel Mirror, using a 0.6 mm exit slit, a 0.40 soller slit, a LiF flat crystal diffracted beam monochromator and a NaI scintillation detector. A detector scan was run using a tube angle of 1° 2θ. Step scans were run from 2 to 35° 2θ, at 0.050 per step, 4 sec per step. A reference quartz standard was used to check instrument alignment. Samples were prepared for analysis by filing a zero background quartz holder.

2. Moisture Balance

Moisture adsorption/desorption data were collected on a VTI SGA-100 moisture balance system, available from VTI Corporation of Hialeah, Fla. For adsorption isotherms, an adsorption range of 5 to 95% relative humidity and a desorption range of 95 to 5% relative humidity in 5% relative humidity increments were used for analysis. The samples were dried at 50° C. prior to analysis. The analyses were conducted at 25° C. Equilibrium criteria used for the analysis were less than 0.001 percent change in 5 minutes with a maximum equilibration time of 1200 minutes if the weight criterion was not met.

In order that this invention be more fully understood, the following examples are set forth. These examples are for the purpose of illustrating embodiments of this invention, and are not to be construed as limiting the scope of the invention in any way. The Compound (1) used in the following examples can be prepared as described in WO 00/59929.

### EXAMPLES

### Example 1

### Preparation of Type A of Compound (1)

A mixture is prepared at about 25° C. using about 28 grams of Compound (1) solid, about 28 ml of water, and about 249 ml of ethanol. The mixture is stirred and heated to at least 70° C., preferably 70-80° C. The Compound (1) solids completely dissolve between 50° C. and 70° C. Separately, a solution is prepared at approximately 25° C. consisting of about 90 volumes of water and about 10 volumes of ethanol. About 271 grams of the water-ethanol solution is added approximately linearly in time over about 2 hours to the Compound (1) solution while maintaining the mixture temperature above about 60° C. and preferably above 70° C. “Type A” Compound (1) begins to crystallize during the water-ethanol addition. When the water-ethanol addition is complete, the resulting crystal slurry is cooled over about 1 hour to between 0° C. and 25° C. and stirred at the final temperature for up to 24 hours. The crystals are filtered and washed with 0-25° C. water, ethanol, or a water-ethanol solution. The wet crystals are dried at temperatures between 10° C. and 100° C., in air or nitrogen atmosphere, at pressures between 1 atm to about 29″ Hg vacuum, to an approximately constant weight. The weight yield is approximately 90% Compound (1) “Type A.”

FIG. 4 shows the Differential Scanning Calorimetry (DSC) thermal curve for the Type A crystals prepared by this ethanol/water process. The thermal curve was obtained using a Perkin Elmer DSC7. The samples were heated from 30° C. to 220° C. at 10° C. per minute, in a sealed pan with a pinhole, using a nitrogen purge flow rate of 25 mL per minute. With reference to these “ethanol/water process” crystals: Type A loses water in the range of ambient on up to ˜100° C., becoming a dehydrated hydrate; the extrapolated onset of melting for the anhydrous phase of Type A is about 186° C., and the endothermic maximum for Type A is at about 198° C.

### Example 2

### Alternative Preparation of Type A of Compound (1)

5.23 g of Compound (Ij Type B are added to 77.86 g of acetonitrile at about 25° C., and the mixture is stirred for about 15 minutes to dissolve Compound (1). The solution is seeded with about 0.059 g of Type A and heated to about 75° C. while stirring. About 3.19 g of water is then added to the solution while maintaining the solution at a temperature of about 75° C. in order to obtain a water concentration of about 4 weight % versus the total solvent components. The solution is then cooled to ambient temperature (about 25° C.) at a rate of about 8° C./hr while stirring. Stirring is continued at ambient temperature for several hours and the resulting crystals of Type A are filtered and dried under ambient air in a vacuum oven. The weight yield is approximately 95% Compound (1) “Type A”.

FIG. 5 shows the Differential Scanning Calorimetry (DSC) thermal curve for the Type A crystals prepared by this acetonitrile process. The thermal curve was obtained using a TA Instruments Q1000 DSC. The samples were heated from 20° C. to 230° C. at 10° C. per minute, in a crimped cup under a nitrogen purge flow rate of approximately 50 mL per minute. With reference to these “acetonitrile process” crystals: The extrapolated onset of melting for the anhydrous phase of Type A is about 199° C., and the endothermic maximum for Type A is at about 208° C.

### Example 3

### Preparation of Type B of Compound (1)

A mixture is prepared at about 25° C. using, for example, about 50 grams of Compound (1) solid and about 200 ml of ethanol. The mixture is stirred and heated until the Compound (1) solids dissolve, which in this example occurs at about 42° C., although when other solvents are used instead of ethanol the dissolution temperature may be up to 700° C. The resulting solution is cooled over a few minutes or as long as desired to between about 0° C. to 25° C. “Type B” Compound (1) begins to crystallize during the cooling. The resulting crystal slurry may be filtered immediately or stirred indefinitely, then filtered. The filtered crystals are washed with 0-25° C. water, ethanol, or a water-ethanol solution. The wet crystals are dried at temperatures between 10° C. and 50° C., in air or nitrogen atmosphere, at pressures between 1 atm to about 29″ Hg vacuum, to an approximately constant weight. The weight yield is approximately 90% Compound (1) “Type B.”

### Example 4

### Alternative Preparation of Type B of Compound (1)

Several micrograms of solid Compound (1) were deposited on to a microscope slide and covered with a glass cover slip. Enough absolute ethanol was introduced under the cover slip using a micro-pipette in order to dissolve the solid Compound (1). The drug substance/solvent solution was allowed to evaporate to dryness at room temperature and subsequently examined under a polarized light microscope for crystalline material. The resulting crystals were subsequently designated as Type B. The experiment was repeated using ˜5 mg of solid Compound (1) in a micro breaker. An XRPD pattern was obtained from the resulting crystals. This material was subsequently designated as Type B.

We claim:

1. A crystalline phase of the following Compound (1): 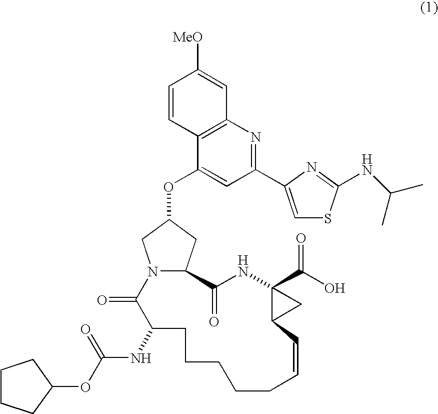
having an X-ray powder diffraction pattern having at least a characteristic peak at 6.9 degrees 2θ (±0.4 degrees 2θ) measured using CuKα radiation, wherein all other peaks in the pattern have less than 75% intensity relative to the peak at 6.9 degrees 2θ (±0.4 degrees 2θ).

2. A crystalline phase of Compound (1) according to claim 1, wherein the crystalline phase further exhibits characteristic peaks at least at 20.9 and 22.7 degrees 2θ (±0.4 degrees 2θ) measured using CuKα radiation.

3. A crystalline phase of Compound (1) according to claim 1, wherein the crystalline phase further exhibits characteristic peaks at least at 16.1, 16.7, 20.9 and 22.7 degrees 2θ (±0.4 degrees 2θ) measured using CuKα radiation.

4. A crystalline phase of Compound (1) according to claim 1, wherein the crystalline phase further exhibits characteristic peaks at least at 8.0, 12.5, 13.9, 14.9, 16.1, 16.7, 17.5, 20.9, 22.7 and 24.1 degrees 2θ (±0.4 degrees 2θ) measured using CuKα radiation.

5. A crystalline phase of Compound (1) according to claim 1, wherein the crystalline phase exhibits an X-ray powder diffraction pattern substantially the same as that shown in FIG. 1 at a relative humidity level of about 30%.

6. A crystalline phase of Compound (1) according to claim 1, wherein the crystalline phase exhibits an X-ray powder diffraction pattern substantially the same as that shown in FIG. 2 at a relative humidity level of about 85%.

7. A crystalline phase of Compound (1) according to claim 1, wherein the crystalline phase has a water adsorption/desorption isotherm substantially the same as that shown in FIG. 3 at 25° C.

8. A crystalline phase of Compound (1) according to claim 1, wherein the crystalline phase has DSC thermal curve substantially the same as that shown in FIG. 4 at a heating rate of 10° C. per minute.

9. A crystalline phase of Compound (1) according to claim 1, wherein the crystalline phase has DSC thermal curve substantially the same as that shown in FIG. 5 at a heating rate of 10° C. per minute.

10. A process for preparing a crystalline phase of Compound (1) according to claim 1, said process comprising the following steps (i) and either (ii)(a) or (ii)(b):
(i) dissolving Compound (1) in an aliphatic alcohol solvent optionally containing water as a co-solvent; and
(ii)(a) adding water, or a mixture of water and an aliphatic alcohol, to the solution obtained in step (i) while maintaining the solution at a temperature above about 55° C.; or
(ii)(b) adding the solution obtained in step (i) to water, or a mixture of water and an aliphatic alcohol, while maintaining the water, or mixture of water and an aliphatic alcohol, at a temperature above about 55° C.

11. A process for preparing a crystalline phase of Compound (1) according to claim 1, said process comprising the following steps:
(i) dissolving or suspending Compound (1) in acetonitrile to form a solution or slurry;
(ii) optionally seeding the solution or slurry obtained in step (i) with Type A;
(iii) heating the solution or slurry to a temperature of at least about 75° C.;
(iv) adding water to the heated solution or slurry obtained in step (iii) while maintaining the solution or slurry at a temperature of at least about 75° C. to obtain a solution or slurry having a water content of about 3 to 5 weight percent; and
(v) slowly cooling the solution or slurry obtained in step (iv).

12. A crystalline phase of the following Compound (1) 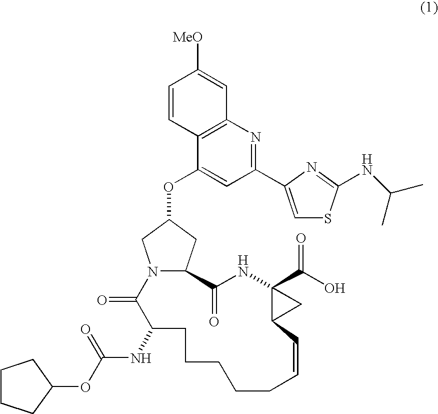
prepared by a process comprising the following steps (i) and either (ii)(a) or (ii)(b):
(i) dissolving Compound (1) in an aliphatic alcohol solvent optionally containing water as a co-solvent; and
(ii)(a) adding water, or a mixture of water and an aliphatic alcohol, to the solution obtained in step (i) while maintaining the solution at a temperature above about 55° C.; or
(ii)(b) adding the solution obtained in step (i) to water, or a mixture of water and an aliphatic alcohol, while maintaining the water, or mixture of water and an aliphatic alcohol, at a temperature above about 55° C.

13. A crystalline phase of the following Compound (1) 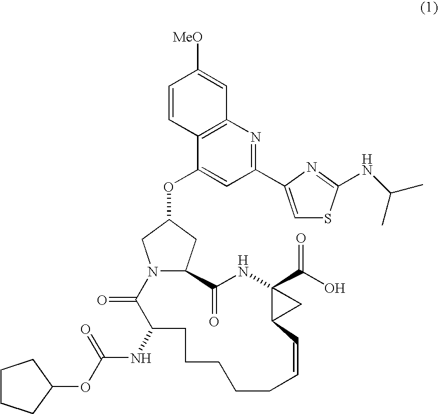
prepared by a process comprising the following steps:
(i) dissolving or suspending Compound (1) in acetonitrile to form a solution or slurry;
(ii) optionally seeding the solution or slurry obtained in step (i) with Type A;
(iii) heating the solution or slurry to a temperature of at least about 75° C.;
(iv) adding water to the heated solution or slurry obtained in step (iii) while maintaining the solution or slurry at a temperature of at least about 75° C. to obtain a solution or slurry having a water content of about 3 to 5 weight percent; and
(v) slowly cooling the solution or slurry obtained in step (iv).

14. A crystalline phase of the following Compound (1): 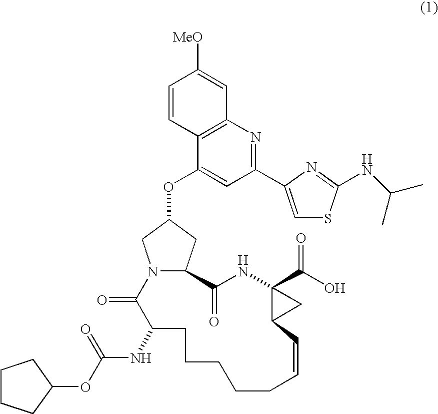
having an X-ray powder diffraction pattern having at least a characteristic peak at 5.4 degrees 2θ (±0.2 degrees 2θ) measured using CuKα radiation at a relative humidity level of about 20% to 40%, and at a temperature of about 20 to 25° C.

15. A crystalline phase of Compound (1) according to claim 14, wherein the crystalline phase further exhibits characteristic at least peaks at 6.7 and 10.9 degrees 2θ (±0.2 degrees 2θ) measured using CuKα radiation at a relative humidity level in the range of about 20% to 40%, and at a temperature of about 20 to 25° C.

16. A crystalline phase of Compound (1) according to claim 14, wherein the crystalline phase further exhibits characteristic peaks at least at 6.7, 10.9, 11.6 and 20.9 degrees 2θ (±0.2 degrees 2θ) measured using CuKα radiation at a relative humidity level in the range of about 20% to 40% and at a temperature of about 20 to 25° C.

17. A crystalline phase of Compound (1) according to claim 14, wherein the crystalline phase exhibits characteristic peaks at least at 5.4, 6.7, 9.4, 10.3, 10.9, 11.6, 13.2 and 20.9 degrees 2θ (±0.2 degrees 2θ) measured using CuKα radiation at a relative humidity level in the range of about 20% to 40% and at a temperature of about 20 to 25° C.

18. A crystalline phase of Compound (1) according to claim 14, wherein the crystalline phase exhibits an X-ray powder diffraction pattern substantially the same as that shown in FIG. 6 at a relative humidity of about 30%.

19. A crystalline phase of Compound (1) according to claim 14, wherein the crystalline phase has a water adsorption/desorption isotherm substantially the same as that shown in FIG. 7 at 25° C.

20. A process for preparing a crystalline phase of Compound (1) according to claim 14, said process comprising:
(i) dissolving Compound (1) in a suitable solvent by heating a mixture of Compound (1) and the solvent; and
(ii) cooling the solution obtained in step (i).

21. A process for preparing a crystalline phase of Compound (1) according to claim 14, said process comprising:
(i) dissolving Compound (1) in an aliphatic alcohol solvent; and
(ii) evaporating the aliphatic alcohol solvent from the solution obtained in step (i).

22. A crystalline phase of the following Compound (1): 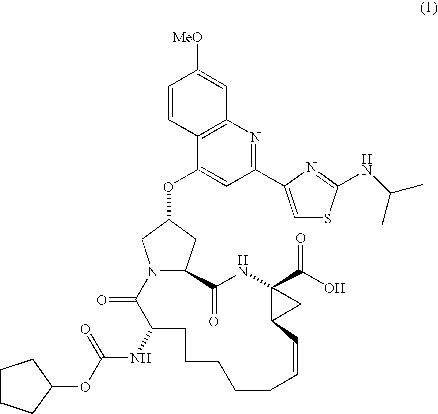
prepared by a process comprising the following steps (i) and (ii), or steps (i)(a) and (ii)(a):
(i) dissolving Compound (1) in a suitable solvent by heating a mixture of Compound (1) and the solvent; and
(ii) cooling the solution obtained in step (i); or
(i)(a) dissolving Compound (1) in an aliphatic alcohol solvent; and
(ii)(a) evaporating the aliphatic alcohol solvent from the solution obtained in step (i).

23. A mixture comprising a crystalline phase of Compound (1) according to claim 1, 12 or 13 and a crystalline phase of Compound (1) according to claim 14 or 22.

24. The following Compound (1): 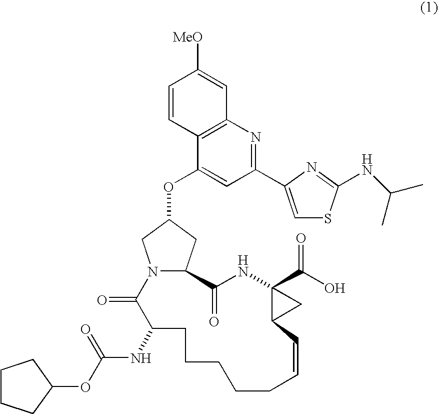
wherein at least 50% of said Compound (1) is present in the form of a crystalline phase of Compound (1) according to claim 1, 12, 13, 14 or 22, or a mixture thereof.

25. A pharmaceutical composition comprising a crystalline phase of Compound (1) according to claim 1, 12, 13, 14 or 22, or a mixture thereof, and at least one pharmaceutically acceptable carrier or diluent.

26. A method of treating HCV infection in a mammal comprising administering to said mammal a therapeutically effective amount of a crystalline phase of Compound (1) according to claim 1, 12, 13, 14 or 22, or a mixture thereof.
